(19) United States
(12) Patent Application Publication
(10) Pub. No.: US 2021/0251512 A1
(43) Pub. Date: Aug. 19, 2021

(54) EVALUATION SYSTEM FOR A VENTILATOR, AND METHOD

(71) Applicant: Loewenstein Medical Technology S.A., Luxembourg (LU)

(72) Inventors: Jan VERHOEVEN, Ettlingen (DE); Benno DOEMER, Ettlingen (DE); Ruediger ALSHUT, Karlsruhe (DE); Matthias SCHWAIBOLD, Karlsruhe (DE)

(21) Appl. No.: 17/173,359

(22) Filed: Feb. 11, 2021

(30) Foreign Application Priority Data
Feb. 13, 2020 (DE) 102020103837.2

(51) Int. Cl. A61B 5/08; A61B 5/00; A61M 16/06; A61M 16/00

(52) U.S. Cl. CPC A61B 5/0803; A61B 5/746; A61M 16/0003; A61M 16/06; A61B 5/743

(57) ABSTRACT

An evaluation system for a ventilator provided for machine ventilation of a patient, comprising an evaluation device which is suitable and configured for recording, over time, a respiration parameter detected by a sensor. The evaluation device is suitable and configured for determining, from a time profile of the respiration parameter, a characteristic number for a respiration stability of the patient, such that the characteristic number provides an assessment of the quality of the ventilation during a period of time.

### CROSS-REFERENCE TO RELATED APPLICATIONS

The present application claims priority under 35 U.S.C. § 119 of German Patent Application No. 102020103837.2, filed Feb. 13, 2020, the entire disclosure of which is expressly incorporated by reference herein.

### BACKGROUND OF THE INVENTION

### 1. Field of the Invention

The present invention relates to an evaluation system for at least one ventilator provided for machine ventilation of a patient, and to a method for operating such an evaluation system. The evaluation system comprises at least one evaluation device which, by way of at least one sensor element, records at least one respiration parameter over time.

### 2. Discussion of Background Information

To be able to monitor the success or the quality of machine ventilation, an evaluation of data is generally carried out which requires greater specialized knowledge on the part of personnel. For an assessment of this kind, it is in most cases necessary to perform time-consuming evaluation of numerous measurement signals or respiration parameters and the profiles of the latter. Monitoring the quality of ventilation is particularly problematic when the patient is being ventilated at home. In contrast to a hospital environment, monitoring of home ventilation can only be done with some difficulty by frequent patient visits.

In view of the foregoing, it would be advantageous to be able to improve the monitoring and assessment of ventilation. In particular, the quality of ventilation should also be able to be reliably estimated by inexperienced users or patients. The monitoring should preferably also be more convenient for the specialist personnel and at the same time more reliable.

### SUMMARY OF THE INVENTION

The present invention provides an evaluation system and a method as set forth in the instant independent claims. Developments and advantageous configurations are the subject matter of the dependent claims. Further advantages and features will become apparent from the general description and from the description of the illustrative embodiments.

The evaluation system according to the invention serves to evaluate at least one respiration parameter, which is detected by one or more sensors during ventilation, and/or other treatment data. The evaluation system is provided for use with at least one ventilator or comprises at least one such ventilator. The ventilator serves in particular for machine ventilation of a patient. According to the invention, this can be understood as controlled ventilation and/or assisted ventilation. The evaluation system comprises at least one evaluation device which is suitable and designed for recording, over time, at least one patient respiration parameter that is detected in particular by at least one sensor element. Here, the evaluation device is suitable and designed for determining, at least from at least one time profile of the at least one respiration parameter, at least one characteristic number for a respiration stability of the patient. The characteristic number thus preferably provides an assessment of the quality of ventilation during a period of time, in particular a defined period of time, for example a period of time of at least about 2 hours or more.

The characteristic number is determined in particular by a statistical evaluation of a breathing pattern recorded over time. The breathing pattern is made available here by the time profile of the at least one respiration parameter. The characteristic number can also be designated as an indicator or index. According to the invention, a characteristic number is understood not only as a number but in particular also as a symbol or the like.

The evaluation system according to the invention affords many advantages. A considerable advantage lies in the provision of the characteristic number by the evaluation system. By looking at the characteristic number, in particular at just a single characteristic number, the user thus obtains a particularly informative and for example statistical assessment of the quality of the ventilation that is performed. Thus, a particularly reliable assessment of the quality of ventilation can be made at one glance, as it were. Moreover, the invention also permits inexperienced users or patients a particularly helpful assessment of the ventilation. Furthermore, the invention also permits safe and convenient monitoring and assessment of ventilation in a domestic environment or in home ventilation, even when the specialist personnel are not present or are only occasionally present. The invention can be used particularly advantageously in telemedicine, for example. Moreover, the invention allows considerable time to be saved, since an often laborious and time-consuming evaluation of respiration parameters over quite long periods of time is no longer necessary, or is necessary only if the characteristic number so dictates.

A considerable advantage is afforded by the provision of a characteristic number for the quality of ventilation or preferably for an assessment of the quality of ventilation over a period of time, since optimal respiration stability without asynchrony or other disturbances is medically advantageous for the patient, and/or the energy consumption or the control outlay of the ventilator is also minimized and therefore the battery time of the ventilator is prolonged or the wear and tear on the electric fan, for example, is minimized.

The evaluation device is preferably suitable and configured for outputting the characteristic number as at least one number and/or at least one graph and/or at least one symbol and/or at least one word and in particular for displaying it with at least one display device. The number can be output with or without units. In all configurations, it is possible that the display device is provided by the evaluation system and/or by the ventilator and/or by other appliances and for example a personal computer or a mobile terminal or the like. It is advantageously possible that the characteristic number is output only by a single number and/or graph and/or only by a single symbol and/or word.

In an advantageous embodiment, the determined characteristic number corresponds to at least one value on a scale or comprises such a value. In particular, one end of the scale is assigned a higher or high and in particular maximum respiration stability and another end of the scale is assigned a lower or low and in particular minimum respiration stability. The evaluation device is preferably suitable and designed for outputting the value and/or the scale with the value positioned thereon, and preferably for indicating it on the display device. A scale between zero and 100 or between one and 100 is preferably provided. It is also possible that a scale of between zero and ten or of between one and ten is provided. Such number ranges permit a particularly clear scale. Scales with other number ranges are also possible.

A dimensionless scale is provided in particular. A scale with units is also possible. The determined characteristic number can also correspond to at least one value range on the scale. It is also possible that the scale and/or the value are defined by at least one word and/or at least one symbol and/or at least one graph or the like. For example, a scale can be provided that ranges, for example, from very good through good and moderate to poor. The symbols provided can be in the form of arrows, for example, which are oriented upward and downward and horizontally. Other symbols are also possible. It is possible and advantageous that the scale is prepared graphically.

In all embodiments, it is particularly advantageous and preferable that the evaluation device is suitable and configured for calculating at least one stability rate from the time profile of the respiration parameter. Preferably, the evaluation device can determine the characteristic number at least from the stability rate. In particular, the characteristic number is calculated from the stability rate by means of at least one assignment function. The stability rate corresponds in particular to a time profile of the stability and/or the variability of the respiration. For example, the stability rate is high when the at least one respiration parameter is stable or when it fluctuates little over time. For example, the stability rate is low when the at least one respiration parameter is unstable or when it fluctuates more over time.

The stability rate can also be formed as a variability rate. It is possible that the stability rate then describes a variability of the respiration or breathing and in particular corresponds to the reciprocal value of the characteristic number. The aforementioned examples are then analogously reversed (a high value corresponds to a high variability or a low stability). Another suitable assignment function can also be provided between stability rate and characteristic number.

In a particularly advantageous embodiment, the evaluation device is suitable and configured for assigning, to at least one time period in which the stability rate exceeds a threshold value for the stability, a characteristic number with a value provided for a maximum respiration stability. It is also possible and advantageous that the evaluation device is suitable and configured for assigning, to at least one time period in which the stability rate exceeds a threshold value for the instability, a characteristic number with a value provided for a minimum respiration stability. The value provided for a maximum respiration stability is in particular the highest value of the scale provided for the characteristic number. The value provided for a minimum respiration stability is in particular the lowest value of the scale provided for the characteristic number.

It is likewise advantageous and preferable that the evaluation device is suitable and configured for assigning, to at least one time period in which the stability rate lies between the threshold value for the stability and the threshold value for the instability, a characteristic number with at least one value. The evaluation device is in particular suitable and configured for calculating the value from the stability rate by means of at least one assignment function and preferably by means of a linear interpolation. Other kinds of assignment functions are also possible.

The evaluation device is preferably suitable and configured for determining the characteristic number during respiration and in particular for updating it at defined time intervals and/or continuously. For example, a characteristic number that indicates low stability can, after quite a long phase of good respiration stability, assume a value that takes account of the improved respiration stability. In particular, the updated characteristic number is averaged from previous characteristic numbers and/or is derived statistically in another way.

It is possible and advantageous that different stability rates and/or characteristic numbers are calculated for at least two or more different respiration parameters. The respective stability rates and/or the respective characteristic numbers are then preferably compared with one another. By way of the comparison, the evaluation device can in particular detect at least one respiratory disturbance and/or at least one respiratory event in the breathing pattern. For example, asynchrony, apnea and/or flow limitations can be detected in this way.

The evaluation device can be suitable and configured for determining the characteristic number using at least one statistical characteristic value for a trend of the time profile of the respiration parameter. Preferably, the statistical characteristic value is an average and particularly preferably a sliding average or comprises at least one such sliding average. Other statistical characteristic values for the trend are also possible.

In an advantageous embodiment, the evaluation device is suitable and configured for determining the characteristic number at least from at least one statistical variation measure for a distribution of values of the respiration parameter in the time profile of the respiration parameter. The statistical variation measure is preferably a variance or comprises at least one such variance. Other suitable statistical variation measures are also possible.

It is possible that the evaluation device is suitable and configured for calculating and in particular outputting at least one trend for a past and/or future development of the characteristic number. For example, it is possible to indicate whether the characteristic number has developed in the direction of higher respiration stability or lower respiration stability in the course of a past time period. Such a trend can be shown, for example, by a symbol and/or a graph and, for example, an arrow.

It is preferable in all embodiments that the at least one respiration parameter used for determining the characteristic number is at least a respiratory rate and/or a tidal volume and/or a respiratory gas flow and/or a respiratory gas pressure or a combination of such parameters. In particular, the evaluation device is suitable and designed for using at least one and preferably at least two or more or all of the aforementioned respiration parameters in order to calculate the characteristic number and/or the stability rate. The evaluation device can also deliberately leave at least one respiration parameter or several respiration parameters out of consideration. The evaluation device can also perform at least one different weighting of certain respiration parameters. The choice or weighting of the respiration parameters can be made, for example, by a type of ventilation set on the ventilator by the evaluation device.

In an advantageous embodiment, the evaluation device is suitable and configured for changing the characteristic number in the direction of poor respiration stability on the basis of at least one of the following causes: asynchrony between ventilator and patient; occurrence of partial or complete intermittent airway obstructions; insomnia; problems caused by mask and/or therapy pressure; apnea; strong formation of secretion; coughing; unstable respiratory drive; periodic breathing; strong leakages.

In an advantageous development, the evaluation device is suitable and designed for executing at least one action if the characteristic number and/or the stability rate lies in at least one defined critical value range for the respiration stability and/or has a trend toward this. It is possible that the action is executed when the characteristic number lies below or above a defined threshold value or trends toward this.

Preferably, the action comprises at least one output of at least one warning and/or at least one alarm and/or at least one action description. The action can be in part acoustic and/or optical and/or haptic. The action can comprise at least one output of the characteristic number in combination with at least one acoustic and/or optical and/or haptic highlighting. It is possible and preferable that the action is executed on at least one appliance coupled to the evaluation device. For example, the action can be executed on a mobile terminal and/or on a personal computer and/or on the ventilator and/or on another appliance.

In an advantageous embodiment, the action comprise at least one actuation of the ventilator. In particular, the actuation concerns at least one respiration parameter. In particular, the ventilator is actuated by the action in such a way that a change of the characteristic number in the direction of a higher and optionally also a lower respiration stability can be achieved. In particular, the ventilator for this purpose regulates at least one respiration parameter to at least one setpoint value stored in the action.

It is advantageous and preferable that at least one critical value range is provided for too high a respiration stability and also that at least one critical value range is provided for too low a respiration stability. This has the advantage that breathing that is too rigid, or too high a respiration stability, can also lead to an action. Thus, breathing disturbances that are associated with high respiration stability can also be indicated by an alarm, for example.

The evaluation device is in particular suitable and configured for outputting the characteristic number in combination with at least one value for at least one respiration parameter. Such a value is preferably processed statistically over time. For example, an average of the respiration parameter can be output in combination with the characteristic number. For example, the characteristic number is output in combination with the respiratory rate and/or the depth of respiration and/or the respiratory gas pressure and/or the respiratory gas flow and/or the tidal volume. The depth of respiration corresponds in particular to the tidal volume or a quotient of the tidal volume and/or a quotient of rate and tidal volume. The characteristic number can also be combined in combination with at least one other characteristic number or with other indices, e.g. for indicating that intervention is required in order to optimize the ventilation, or because of an acute deterioration in the illness.

It is particularly preferable and advantageous that the evaluation device is suitable and configured for actuating the ventilator according to the determined characteristic number, and preferably for adapting at least one control variable of the ventilator in order in particular to obtain a change of the characteristic number in the direction of a higher respiration stability. In particular, the evaluation device and the ventilator are operatively connected. In particular, the evaluation device can control or regulate the ventilator according to the characteristic number and, for example, can adjust this to a setpoint value of at least one respiration parameter.

In particular, the evaluation device preferably is suitable and configured for achieving a higher respiration stability, or for achieving a characteristic number representing a higher respiration stability, by adapting the respiratory gas pressure and/or the respiratory gas flow. Alternatively or in addition, the evaluation device can also for this purpose adapt at least one of the following parameters: inspiratory pressure level; expiratory pressure level; trigger sensitivity; inspiration duration; expiration duration; steepness of the pressure transitions between the pressure levels.

The evaluation device is in particular suitable and configured for determining and/or outputting the characteristic number only after a defined minimum duration of the ventilation and/or for taking account of an action. In particular, the evaluation device is suitable and configured for outputting the characteristic number together with at least one warning as regards undershooting of the minimum duration and/or together with at least one warning as regards undershooting of the minimum duration for an action.

In particular, the minimum duration comprises at least about two hours and preferably at least about four hours and particularly preferably at least about six hours. The minimum duration can also be at least about eight hours or at least about ten hours or also about 12 hours or more. A minimum duration of about half an hour or about one hour or about one and a half hours is also possible. Particularly preferably, the minimum duration comprises at least one night sleep phase of the patient. The night sleep phase preferably comprises at least one NREM phase and/or REM phase. In particular, the night sleep phase is at least about one hour and preferably at least about two hours and particularly preferably at least about four hours or more.

In an advantageous embodiment, the evaluation device is suitable and configured for detecting an acute exacerbation if the characteristic number indicates a rise of the respiration stability of the patient, in particular above a defined rate of rise, and the detected respiration parameter at the same time indicates a rising respiratory rate and shallower breathing. In particular, as a consequence of such a detection, at least one action is executed and for example a warning and/or an alarm is output and/or an action instruction is output. In particular, for the detection of such an event, at least two or more respiration parameters are taken into consideration and evaluated.

The evaluation system can comprise at least one ventilator. The ventilator is in particular suitable and configured to be operated in the above-described manner and in particular to be actuated by the evaluation device. In particular, the ventilator is designed for ventilation in the form of CPAP and/or APAP. In particular, the evaluation device is suitable and configured for determining the characteristic number during CPAP and/or APAP and/or another machine ventilation. In particular, the ventilator is suitable and configured for generating at least one defined respiratory gas flow for ventilation by means of at least one ventilation device.

In particular, the evaluation system comprises at least one sensor element. The sensor element can be made available by the ventilator. In particular, the ventilator has a sensor device which serves to control or regulate the ventilation. For example, at least one flow sensor and/or pressure sensor may be provided. The evaluation system can have further sensors for detecting respiration parameters.

The method according to the invention serves for operating an evaluation system as described above. Such a method also achieves the aforementioned object in a particularly advantageous manner.

In particular, the method is such that the above-described actions of the evaluation device are executed. In particular, the above-described evaluation system is suitable and configured for being operated by the method according to the invention. In the context of the present invention, provision is made in particular that the evaluation device is suitable and configured for implementation of the method features or steps described herein.

In particular, at least one algorithm by which the characteristic number can be calculated from one or more respiration parameters is stored in the evaluation device. In particular, the evaluation device comprises at least one electronic computer unit.

It is possible that the evaluation system is formed separately from the ventilator. For example, the evaluation device is for this purpose accommodated in a housing separate from the ventilator and can be coupled to the latter by wires and/or wirelessly via at least one interface.

The evaluation system can comprise at least one (dedicated) display device and/or can use a display device of the ventilator in order to output the characteristic number and/or other information. The display device of the evaluation system can be supplemented by the display device of the ventilator. The evaluation system can also be able to be coupled to at least one computer and/or at least one mobile terminal or the like, in order there to output the characteristic number and/or other information. The evaluation system can use such an appliance as a display device.

In particular, the at least one respiration parameter is detected during ventilation. Preferably, at least two or at least three respiration parameters and in particular a multiplicity of respiration parameters are detected by the sensor means during ventilation. It is also possible that further respiration parameters are detected by external sensor units and recorded over time and in particular used for the determination of the characteristic number. Such further respiration parameters can be, for example, pulse rate, oxygen content of the blood, CO₂ content of the blood, blood pressure, body temperature, activity and/or other parameters. If, for example, the patient is active and is using his mobile ventilator on a wheelchair, etc., a high variability in this phase is assessed differently than when the body is at rest during the night.

The respiration parameter detected by the sensor element can be derived directly and/or indirectly from at least one sensor variable. In all embodiments, it is preferable that at least two or at least three respiration parameters and preferably a multiplicity of respiration parameters are detected and are taken into consideration for the determination of the characteristic number.

### BRIEF DESCRIPTION OF THE DRAWINGS

Further advantages and features of the present invention will become clear from the description of illustrative embodiments, which are explained below with reference to the accompanying drawings, in which:

FIG. 1 shows a purely schematic and perspective view of an evaluation system according to the invention;

FIG. 2 shows a highly schematic graph with profiles of respiration parameters in order to explain the determination of a characteristic number in the invention; and

FIGS. 3-7 show further highly schematic graphs with profiles of respiration parameters in order to explain the determination of the characteristic number in the invention.

### DETAILED DESCRIPTION OF EXEMPLARY EMBODIMENTS

The particulars shown herein are by way of example and for purposes of illustrative discussion of the embodiments of the present invention only and are presented in the cause of providing what is believed to be the most useful and readily understood description of the principles and conceptual aspects of the present invention. In this regard, no attempt is made to show details of the present invention in more detail than is necessary for the fundamental understanding of the present invention, the description in combination with the drawings making apparent to those of skill in the art how the several forms of the present invention may be embodied in practice.

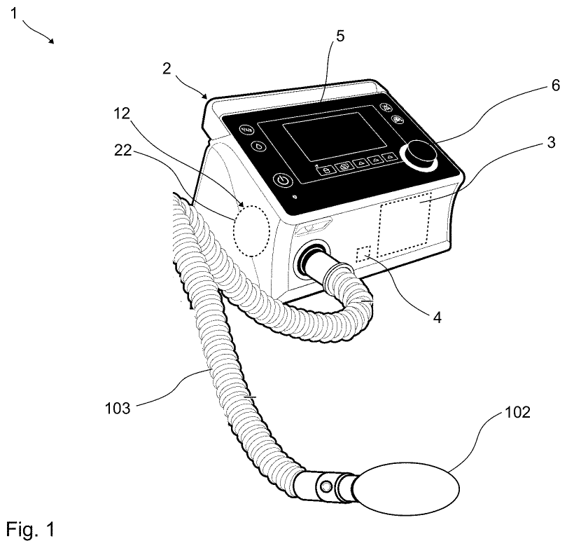

FIG. 1 shows an evaluation system 1 according to the invention, with an evaluation device 3 for assessing the respiration stability during machine ventilation with a ventilator 2. The ventilator 2 is here part of the evaluation system 1 and provides the housing of the evaluation device 3. The evaluation device 3 can alternatively also be arranged as a separate device outside the ventilator 2. The system 1 shown here is operated by the method according to the invention. The evaluation system 1 is designed here as a home ventilator. However, the evaluation system 1 can also be used for ventilation in a hospital environment.

In the interior of its housing, the ventilator 2 here has a ventilation device 12 which is equipped with a fan 22 for generating a respiratory gas flow. The respiratory gas flow is supplied to the patient via a tubing 103 which is coupled to the ventilation device 12 and which has a breathing mask 102.

Alternatively to the breathing mask 102, other patient interfaces can also be used. In addition to or as an alternative to the fan 22, a compressed gas source can also be provided.

The ventilator 2 here comprises a display device 5 and an operating device 6. Combinations of operating device 6 and display device 5 can also be provided, for example in the form of a touch-sensitive display surface or a touch screen. Here, the display device 5 also serves to show information from the evaluation device 3. However, the evaluation device 3 can also output its information on further display devices not shown here, e.g. on a computer display or a tablet or smartphone or the like.

The ventilation device 12 is here operatively connected to a sensor means 4 which has one or more sensors for detecting respiration parameters and, if appropriate, further variables characteristic of the respiration. For example, the sensor means 4 comprises a pressure sensor (not shown in detail here) which detects the pressure conditions of the respiratory gas flow. The sensor means 4 is also operatively connected here to the evaluation device 3, such that the detected variables can also be at least partially processed by the evaluation device 3. The evaluation device 3 can also comprise its own sensor means 4.

The ventilation device 12 here comprises a control device which is arranged in a concealed fashion inside the housing and which actuates the fan 22. For example, CPAP ventilation or APAP ventilation can be carried out. The ventilation device 12 is switched, for example, to a defined respiratory gas flow and/or a respiratory gas pressure for ventilation. The control device 12 can make available a required minimum pressure and/or can compensate for pressure fluctuations that are caused by the breathing activity of the user. For example, the control device 12 detects, by way of the sensor means 4, the current pressure in the breathing mask 102 and regulates the power of the fan 22 accordingly, until a desired respiratory pressure is present.

Moreover, the ventilation device 12 can also be actuated here by the evaluation device 3. For this purpose, the evaluation device 3 is operatively connected to the control device.

In order to assess the quality of the ventilation, the evaluation device 3 detects, by way of the sensor means 4, a plurality of respiration parameters during ventilation and records these over time. From the time profile of the respiration parameters, a characteristic number for a respiration stability of the patient is then determined and shown on the display device 5. Looking at the characteristic number, the user thus obtains an assessment of the quality of the ventilation.

The calculation of the respiration stability is done here by statistical evaluation of the breathing pattern (e.g. in CPAP or APAP mode). During ventilation, it is possible to intervene strongly in the breathing pattern. Ideally, the breathing pattern would be completely rigid in machine ventilation. In assisted breathing, a certain variability is possible and desired by the patient. However, in the course of ventilation over a long period of time, preferably including at least part of the night sleep, sufficiently long phases with stable breathing should be achieved with a good ventilation setting. If this is not the case, it indicates that the ventilation is not optimally set.

The calculated indicator, preferably between 0 and 100, for the respiration stability thus serves as an indication/base for an alarm being triggered when the ventilation needs to be optimized or when there is an acute deterioration in the condition of the patient. A consistently low respiration stability can be caused, for example, by: asynchrony between ventilator (2) and patient; occurrence of partial or complete airway obstructions; insomnia; problems caused by mask (102) and/or therapy pressure; apnea; strong formation of secretion; coughing; unstable respiration drive; periodic breathing; strong leakages.

The value of the characteristic number or of the respiration stability indicator permits rapid orientation on whether further analysis of the patient is required. In combination with further measured parameters such as AHI or asynchrony events, it is possible either to confirm or rule out individual causes of unstable respiration. It is thus preferably possible for the system 1 to automatically adapt respiration settings, such as inspiratory pressure level, expiratory pressure level, trigger sensitivity, inspiration duration, expiration duration, or steepness of the pressure transitions between the pressure levels, and to measure the success of this via an improvement of the respiration stability. Moreover, a warning of an acute deterioration in symptoms/exacerbation can be output, preferably in combination with further parameter such as respiratory rate or depth of respiration (tidal volume, or quotient of frequency and tidal volume). Possible rules: increasing rate, shallower breathing and more rigid breathing (increase of respiration stability) can be an indicator of an acute exacerbation.

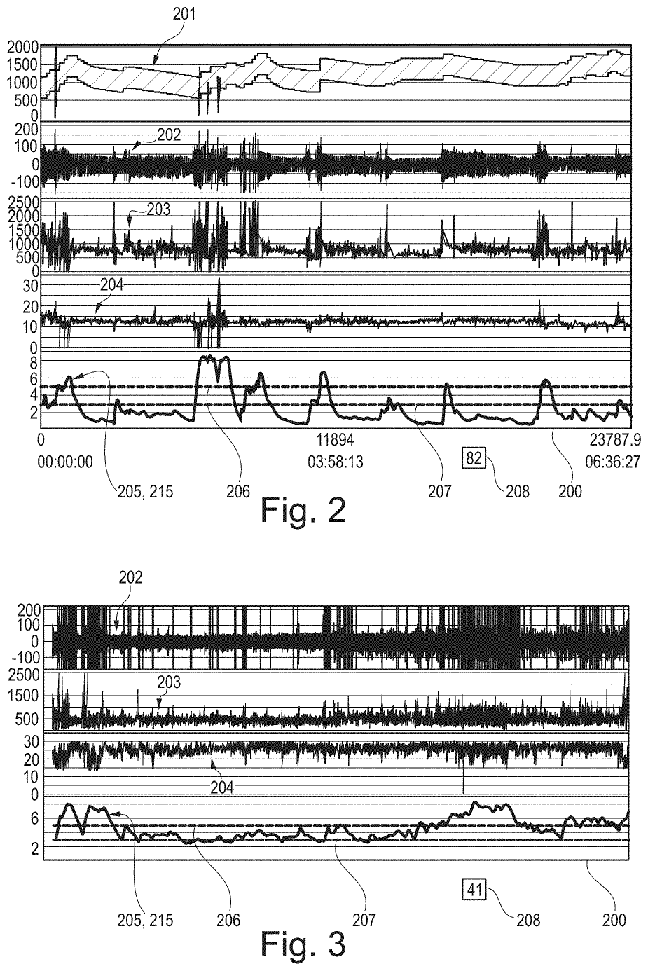

FIG. 2 shows an example of the determination and output of a characteristic number 208, and the respiration parameters forming the basis of its calculation. The respiration parameters were detected here by the sensor means 4 during ventilation and were recorded as profiles over time 200. The measurement period here is 6 h 30 min.

The respiration parameters are here the respiratory gas pressure 201, the respiratory gas flow 202, the tidal volume 203, and the respiratory rate 204. From their profiles, a stability rate 205 was determined which, in the example shown here, is designed as a variability rate 215 and describes the breathing variability. The variability rate 215 thus assumes a higher value when the breathing becomes more variable or more unstable, and vice versa. Such a variability rate 215 can correspond, for example, to the reciprocal value of the desired characteristic number 208.

If the value of the variability rate 215 is high, the respiratory flow/tidal volume/respiratory rate fluctuates greatly between individual breaths. If the value is low, the breathing is regular.

Here, the two lines along the stability rate 205 and the variability rate 215 mark a threshold value 207 for the stability and a threshold value 206 for the instability. Periods in which the curve 215 lies below the lower line 207 are classed as fully stable. The characteristic number 208 then assumes the maximum value provided for the assessment (e.g. score of 100).

Periods in which the curve 215 lies above the upper line 206 are classed as fully unstable. The characteristic number 208 then assumes the minimum value provided for the assessment (e.g. score of 0).

Between the two threshold values 206 and 207, a linear interpolation is performed here between 0 and 100. Thus, an average stability score of 82 is obtained for the respiration of the patient shown here by way of example as characteristic number 208. This is a good value; the breathing is regular over long phases.

For example, in order to determine the stability rate 205 or variability rate 215 from detected respiration parameters, the following formula can be stored in the evaluation device 3 and used: “Variability rate=sliding_average_over_2 min (sum ((respiration_parameter_current_breath−respiration_parameter average_over_2 min)/respiration_parameter average_over_2 min)*100)”.

For example, to derive the characteristic number 208 from the stability rate 205 or variability rate 215, the following formula can be stored in the evaluation device 3 and used: “Characteristic_number_entire_measurement=100-average entire_measurement (variability rate)”.

The evaluation device 3 can be designed here such that different stability rates 205 or variability rates 215 can be calculated and compared for two or more respiration parameters. For example, if the respiratory rate fluctuates more than the tidal volume in one phase, this points to asynchrony. If the volume fluctuates more than the rate, this points to apnea or flow limitations.

FIG. 3 shows a further example of the determination and output of a characteristic number 208 and the respiration parameters on which the calculation thereof is based. Here, the patient has many instances of apnea/pauses in breathing. These are indicated here by the vertical lines in the respiratory flow 202. Here, the breathing is never entirely stable at all. As characteristic number 208, an average score of 41 is obtained for the respiration of the patient shown here by way of example.

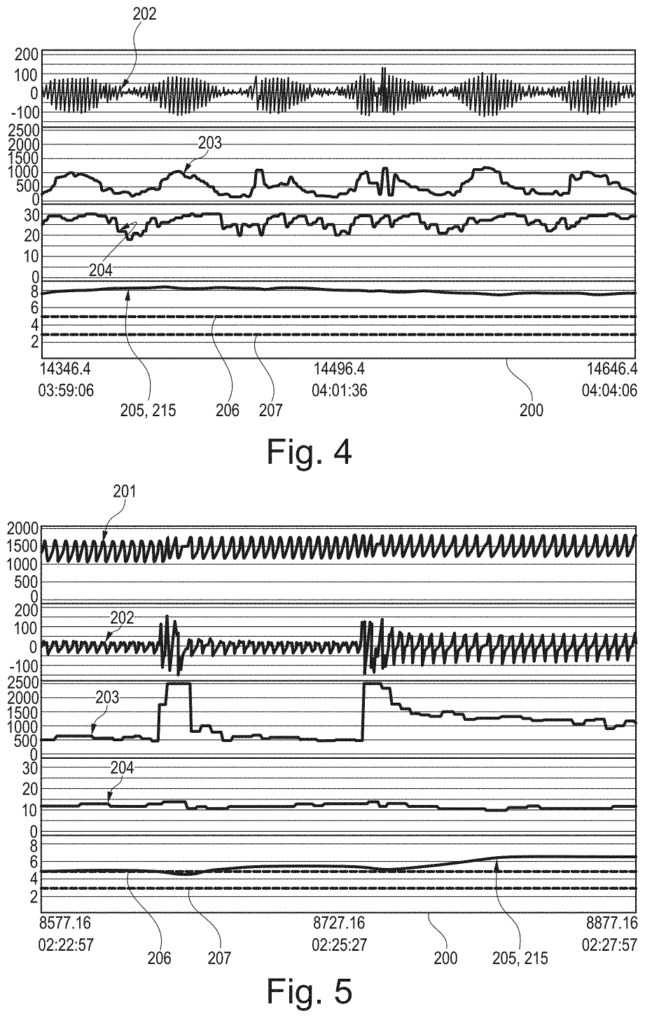

FIGS. 4 to 7 show examples of situations that may lead to respiratory unrest. There are therefore significantly increased variability rates 215 here. The variability rates 215 here in each case describe, as before in FIGS. 2 and 3 also, the breathing variability.

FIG. 4 shows a breathing pattern with apnea.

FIG. 5 shows a breathing pattern with flow limitations, which are interrupted by arousals. The flow limitations can be due to increased resistance of the upper airways.

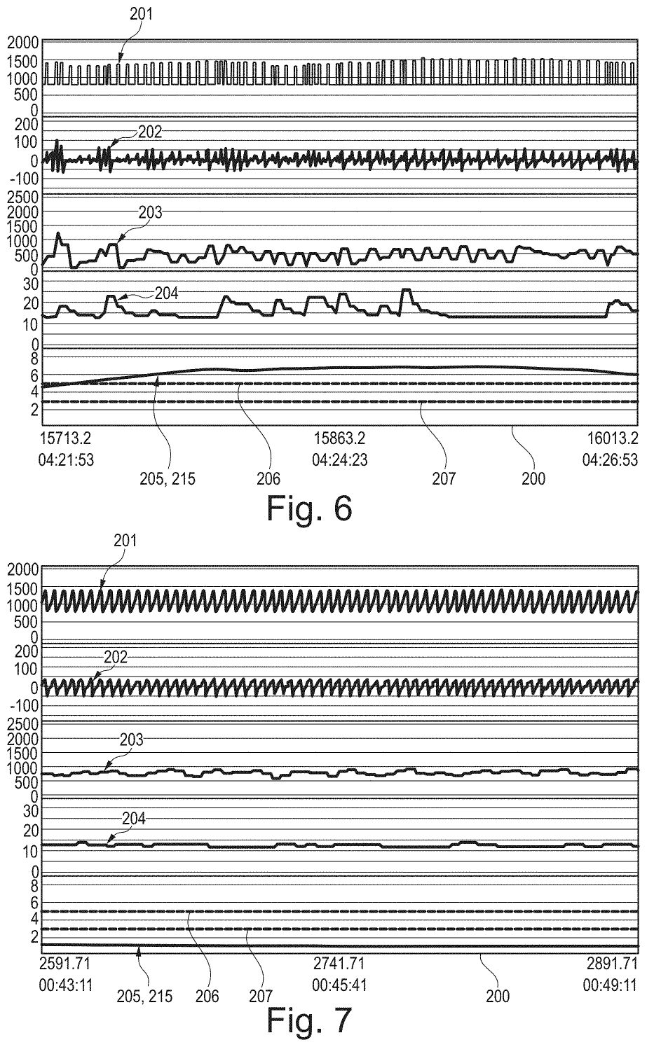

FIG. 6 shows a breathing pattern with considerable asynchrony, such that here the appliance 1 and the patient work against each other.

FIG. 7 shows a breathing pattern with particularly smooth respiration. The stability rate 205, here formed as variability rate 215, lies permanently below the threshold value 207 here, such that the characteristic number 208 here assumes a maximum possible value.

To sum up, the present invention provides:
1. An evaluation system for at least one ventilator provided for machine ventilation of a patient, wherein the evaluation system comprises at least one evaluation device which is suitable and configured for recording, over time, at least one respiration parameter detected by at least one sensor, the evaluation device being suitable and configured for determining at least, from at least one time profile of the respiration parameter, at least one characteristic number for a respiration stability of the patient, such that the characteristic number provides an assessment of a quality of the ventilation during a period of time.
2. The evaluation system of item 1, wherein the evaluation device is suitable and configured for outputting the characteristic number as at least one number and/or graph and/or at least one symbol and/or word and preferably for displaying it on at least one display device.
3. The evaluation system of any one of items 1 or 2, wherein the determined characteristic number corresponds to at least one value on a scale, and wherein one end of the scale is assigned a higher or maximum respiration stability and another end of the scale is assigned a lower or minimum respiration stability, the evaluation device being suitable and configured for outputting the value and/or the scale with the at least one value positioned thereon and preferably for displaying it on at least one display device.
4. The evaluation system of any one of the preceding items, wherein the evaluation device is suitable and configured for calculating at least one stability rate from a time profile of the respiration parameter and for determining the characteristic number at least from the stability rate.
5. The evaluation system of item 4, wherein the evaluation device is suitable and configured for assigning, to a time period in which a stability rate exceeds a threshold value for the stability, a characteristic number with a value provided for a maximum respiration stability, and/or for assigning, to a time period in which the stability rate exceeds a threshold value for the instability, a characteristic number with a value provided for a minimum respiration stability.
6. The evaluation system of item 5, wherein the evaluation device is suitable and configured for assigning, to a time period in which the stability rate lies between the threshold value for the stability and the threshold value for the instability, a characteristic number with one value, and for calculating the value from the stability rate by at least one assignment function and preferably by linear interpolation.
7. The evaluation system of any one of the preceding items, wherein the evaluation device is suitable and configured for determining the characteristic number during respiration and for updating it at defined time intervals and/or continuously, such that the characteristic number takes at least partial account of past time periods and of the values assigned to these time periods for the characteristic number.
8. The evaluation system of any one of the preceding items, wherein the evaluation device is suitable and configured for determining the characteristic number using at least one statistical characteristic value for a trend of the time profile of the respiration parameter, and wherein the statistical characteristic value is preferably an average and particularly preferably a sliding average or at least comprises such a sliding average.
9. The evaluation system of any one of the preceding items, wherein the evaluation device is suitable and configured for determining the characteristic number at least from at least one statistical variation measure for a distribution of values in a time profile of the respiration parameter, and wherein the statistical variation measure is preferably a variance or at least comprises such a variance.
10. The evaluation system of any one of the preceding items, wherein the evaluation device is suitable and configured for calculating and outputting a trend for a past and/or future development of the characteristic number.
11. The evaluation system of any one of the preceding items, wherein the respiration parameter used for determining the characteristic number is at least a respiratory rate and/or a tidal volume and/or a respiratory gas flow and/or a respiratory gas pressure.
12. The evaluation system of any one of the preceding items, wherein the evaluation device is suitable and configured for changing the characteristic number in a direction of poor respiration stability based on at least one of the following causes: asynchrony between ventilator and patient; occurrence of partial or complete intermittent airway obstructions; insomnia; problems caused by mask and/or therapy pressure; apnea; strong formation of secretion; coughing; unstable respiratory drive; periodic breathing; strong leakages.
13. The evaluation system of any one of the preceding items, wherein the evaluation device is suitable and configured for executing at least one action if the characteristic number lies in at least one defined critical value range for the respiration stability and/or has a trend toward this, and wherein the action comprises an output of at least one warning and/or alarm and/or an action description.
14. The evaluation system of item 13, wherein at least one critical value range is provided for too high a respiration stability and at least one critical value range is provided for too low a respiration stability, such that breathing that is too rigid can also lead to an action.
15. The evaluation system of any one of the preceding items, wherein the evaluation device is suitable and configured for outputting the characteristic number in combination with at least one value for at least one respiration parameter, for example respiratory rate and/or depth of respiration, the at least one value being preferably processed statistically over time.
16. The evaluation system of any one of the preceding items, wherein the evaluation device is suitable and configured for actuating the ventilator according to the determined characteristic number, and in particular for adapting at least one control variable of the ventilator in order to obtain a change of the characteristic number in a direction of a higher respiration stability.
17. The evaluation system of item 16, wherein the evaluation device is suitable and configured for achieving a higher respiration stability by adapting a respiratory gas pressure and/or a respiratory gas flow and/or by adapting at least one of the following parameters: inspiratory pressure level; expiratory pressure level; trigger sensitivity; inspiration duration; expiration duration; steepness of the pressure transitions between the pressure levels.
18. The evaluation system of any one of the preceding items, wherein the evaluation device is suitable and configured for outputting the characteristic number only after a defined minimum duration of the ventilation and/or for taking account of an action, and/or wherein the evaluation device is suitable and configured for outputting the characteristic number together with at least one warning as regards undershooting of a minimum duration and/or together with at least one warning as regards undershooting of a minimum duration for an action, the minimum duration comprising at least two hours and/or at least one night sleep phase of the patient.
19. The evaluation system of any one of the preceding items, wherein the evaluation device is suitable and configured for detecting an acute exacerbation if the characteristic number indicates a rise of a respiration stability of the patient and a detected respiration parameter at the same time indicates a rising respiratory rate and shallower breathing.
20. The evaluation system of any one of the preceding items, wherein the system comprises at least one ventilator.
21. A method for operating the evaluation system of any one of the preceding items.

### LIST OF REFERENCE NUMERALS

1 evaluation system
2 ventilator
3 evaluation device
4 sensor means
5 display device
6 operating device
12 ventilation device
22 fan
102 breathing mask
103 tubing
200 time
201 pressure
202 flow
203 volume
204 rate
205 stability rate
206 threshold value
207 threshold value
208 characteristic number
215 variability rate

What is claimed is:

1. An evaluation system for at least one ventilator provided for machine ventilation of a patient, wherein the evaluation system comprises at least one evaluation device which is suitable and configured for recording, over time, at least one respiration parameter detected by at least one sensor, the evaluation device being suitable and configured for determining at least, from at least one time profile of the respiration parameter, at least one characteristic number for a respiration stability of the patient, such that the characteristic number provides an assessment of a quality of the ventilation during a period of time.

2. The evaluation system of claim 1, wherein the evaluation device is suitable and configured for outputting the characteristic number as at least one number and/or graph and/or at least one symbol and/or word.

3. The evaluation system of claim 1, wherein the determined characteristic number corresponds to at least one value on a scale, one end of the scale being assigned a higher or maximum respiration stability and another end of the scale being assigned a lower or minimum respiration stability, and wherein the evaluation device is suitable and configured for outputting the value and/or the scale with the at least one value positioned thereon.

4. The evaluation system of claim 1, wherein the evaluation device is suitable and configured for calculating at least one stability rate from a time profile of the respiration parameter and for determining the characteristic number at least from the at least one stability rate.

5. The evaluation system of claim 4, wherein the evaluation device is suitable and configured for assigning, to a time period in which a stability rate exceeds a threshold value for the stability, a characteristic number with a value provided for a maximum respiration stability, and/or for assigning, to a time period in which the stability rate exceeds a threshold value for the instability, a characteristic number with a value provided for a minimum respiration stability.

6. The evaluation system of claim 5, wherein the evaluation device is suitable and configured for assigning, to a time period in which the stability rate lies between a threshold value for the stability and a threshold value for the instability, a characteristic number with one value, and for calculating the value from the stability rate by at least one assignment function.

7. The evaluation system of claim 1, wherein the evaluation device is suitable and configured for determining the characteristic number during respiration and for updating it at defined time intervals and/or continuously, such that the characteristic number takes at least partial account of past time periods and of values assigned to these time periods for the characteristic number.

8. The evaluation system of claim 1, wherein the evaluation device is suitable and configured for determining the characteristic number using at least one statistical characteristic value for a trend of a time profile of the respiration parameter.

9. The evaluation system of claim 1, wherein the evaluation device is suitable and configured for determining the characteristic number at least from at least one statistical variation measure for a distribution of values in a time profile of the respiration parameter.

10. The evaluation system of claim 1, wherein the evaluation device is suitable and configured for calculating and outputting a trend for a past and/or future development of the characteristic number.

11. The evaluation system of claim 1, wherein the respiration parameter used for determining the characteristic number is at least a respiratory rate and/or a tidal volume and/or a respiratory gas flow and/or a respiratory gas pressure.

12. The evaluation system of claim 1, wherein the evaluation device is suitable and configured for changing the characteristic number in a direction of poor respiration stability based on at least one of the following causes: asynchrony between ventilator and patient; occurrence of partial or complete intermittent airway obstructions; insomnia; problems caused by mask and/or therapy pressure; apnea; strong formation of secretion; coughing; unstable respiratory drive; periodic breathing; strong leakages.

13. The evaluation system of claim 1, wherein the evaluation device is suitable and configured for executing at least one action if the characteristic number lies in at least one defined critical value range for a respiration stability and/or has a trend toward this, and wherein the action comprises an output of at least one warning and/or alarm and/or an action description.

14. The evaluation system of claim 13, wherein at least one critical value range is provided for too high a respiration stability and at least one critical value range is provided for too low a respiration stability, such that breathing that is too rigid can also lead to an action.

15. The evaluation system of claim 1, wherein the evaluation device is suitable and configured for outputting the characteristic number in combination with at least one value for at least one respiration parameter.

16. The evaluation system of claim 1, wherein the evaluation device is suitable and configured for actuating the ventilator according to the determined characteristic number.

17. The evaluation system of claim 16, wherein the evaluation device is suitable and configured for achieving a higher respiration stability by adapting a respiratory gas pressure and/or a respiratory gas flow and/or by adapting at least one of the following parameters: inspiratory pressure level; expiratory pressure level; trigger sensitivity; inspiration duration; expiration duration; steepness of the pressure transitions between the pressure levels.

18. The evaluation system of claim 1, wherein the evaluation device is suitable and configured for outputting the characteristic number only after a defined minimum duration of the ventilation and/or for taking account of an action, and/or wherein the evaluation device is suitable and configured for outputting the characteristic number together with at least one warning as regards undershooting of a minimum duration and/or together with at least one warning as regards undershooting of the minimum duration for an action, the minimum duration comprising at least two hours and/or at least one night sleep phase of the patient.

19. The evaluation system of claim 1, wherein the evaluation device is suitable and configured for detecting an acute exacerbation if the characteristic number indicates a rise of a respiration stability of the patient and a detected respiration parameter at the same time indicates a rising respiratory rate and shallower breathing.

20. The evaluation system of claim 1, wherein the system comprises at least one ventilator.
